(19) United States
(12) Patent Application Publication
(10) Pub. No.: US 2011/0320010 A1
(43) Pub. Date: Dec. 29, 2011

(54) Prosthetic Devices Having a Universal Socket Design and Methods of Making and Using the Same

(76) Inventor: Ha Van Vo, Macon, GA (US)

(21) Appl. No.: 13/254,461

(22) PCT Filed: Mar. 5, 2010
(86) PCT No.: PCT/US10/26435

§ 371 Date: Sep. 2, 2011

Related U.S. Application Data
(60) Provisional application No. 61/157,767, filed on Mar. 5, 2009.
(60) Provisional application No. 61/183,095, filed on Jun. 2, 2009.

(51) Int. Cl. A61F 2/78

(52) U.S. Cl. 623/33

(57) ABSTRACT

A prosthetic device (10) comprising: a universal socket (11) operatively adapted and sized to receive a variety of stump sizes, said universal socket comprising: a first socket open end (25) sized to receive a user stump, a second socket end (27) opposite the first socket end, at least two differently sized socket regions (L, M, S) positioned between the first socket open end and the second socket end, said at least two differently sized socket regions comprising an upper socket region proximate the first socket open end and a lower socket region positioned between the upper socket region and the second socket end, wherein the upper socket region has an upper region cross-sectional area, the lower socket region has an lower region cross-sectional area, and the upper region cross-sectional area is greater than the lower region cross-sectional area.

This application is being filed as a PCT International Patent Application in the name of The Corporation of Mercer University, a U.S. corporation, claiming priority to (i) U.S. Provisional Patent Application Ser. No. 61/157,767 filed on 5 Mar. 2009 and (ii) U.S. Provisional Patent Application Ser. No. 61/183,095 filed on 2 Jun. 2009, both of which are entitled “PROSTHETIC DEVICES HAVING A UNIVERSAL SOCKET DESIGN AND METHODS OF MAKING AND USING THE SAME.”

### TECHNICAL FIELD

The present invention relates to prosthetic devices having a universal socket design. The present invention also relates to methods of making and methods of using prosthetic devices having a universal socket design.

### BACKGROUND

A customized socket for an amputee costs from about $3,500 to $7,000. In addition, the distal attachments of the socket include a pylon, ankle, and foot for below knee amputation; these components cost about $500 to $3,000. For an above-knee amputation, the distal attachments of the socket include the knee, pylon, ankle, and foot; these components cost about $1,200 to $6,000. Total cost for a below-knee amputation prosthesis is from about $4,000 to $10,000 and about $4,700 to $13,000 for an above-knee amputation prosthesis in the USA (source—Hanger Prosthetics & Orthotics Inc. (Macon, Ga.)).

In third world countries, especially post-war countries such as Vietnam, Korea, Afghanistan, Cambodia, Laos, Iraq, and Haiti, amputees can not afford the above-mentioned prices for prosthesis. What is needed in the art is a relatively inexpensive, effective prosthesis.

### SUMMARY

The present invention addresses some of the difficulties and problems discussed above by the discovery of an inexpensive prosthesis comprising a universal and adjustable design that can fit in to a wide range of stumps for above or below the knee amputees. In some exemplary embodiments, the disclosed universal prosthetic socket comprises at least three noticeably distinct pre-made socket sizes (e.g., large, medium, and small) positioned along a height of the universal prosthetic socket so as to accommodate various sizes of stumps. In other exemplary embodiments, the disclosed universal prosthetic socket comprises a gradually tapering configuration that enables the socket to accommodate various sizes of stumps. In addition, the disclosed universal prosthetic sockets may be provided in a variety of overall sizes (e.g., large, medium, small, very small) to accommodate amputees ranging from relatively large adults to children.

Accordingly, in one exemplary embodiment, the present invention is directed to prosthetic devices. The disclosed prosthetic devices comprise a universal socket operatively adapted and sized to receive a variety of stump sizes, wherein the universal socket comprises a first socket open end sized to receive a user stump, a second socket end opposite the first socket end, at least two differently sized socket regions positioned between the first socket open end and the second socket end, the at least two differently sized socket regions comprising an upper socket region proximate the first socket open end and a lower socket region positioned between the upper socket region and the second socket end, wherein the upper socket region has an upper region cross-sectional area, the lower socket region has an lower region cross-sectional area, and the upper region cross-sectional area is greater than the lower region cross-sectional area.

In some embodiments, the at least two differently sized socket regions comprise socket regions, wherein each socket region has a side wall component that extends substantially vertically, and each side wall component of a given socket region is separated from an adjacent socket region by a side wall component that extends both horizontally, and typically both horizontally and vertically. As discussed below, this configuration is referred to herein as a “non-tapered configuration.” In other embodiments, the at least two differently sized socket regions comprise socket regions, wherein each socket region has a side wall component that appears as an extension of the side wall of an adjacent socket region. As discussed below, this configuration is referred to herein as a “tapered configuration.”

The disclosed prosthetic devices may comprise a number of additional features as described below and in the claims. In addition, the disclosed prosthetic devices may comprise a number of components, other than the universal socket, that enable customized fitting of a given prosthetic device to a specific user. Components suitable for use with the universal socket include, but are not limited to, one or more stump cushions for filling a desired amount of space with a given universal socket between a user's stump end and the second socket end; one or more rings or ring portions; a knee joint for above-knee prosthetics; and any combination thereof.

The present invention is further directed to a method of making prosthetic devices. The present invention is even further directed to a method of using prosthetic devices.

These and other features and advantages of the present invention will become apparent after a review of the following detailed description of the disclosed embodiments and the appended claims.

### BRIEF DESCRIPTION OF THE FIGURES

FIG. 1A depicts a view of an exemplary prosthetic device of the present invention;

FIG. 1B depicts exemplary stump cushions for use in the exemplary prosthetic device shown in FIG. 1A;

FIG. 1C depicts exemplary ring portions for use in the exemplary prosthetic device shown in FIG. 1A;

FIG. 1D depicts a single exemplary stump cushion for use in the exemplary prosthetic device shown in FIG. 1A;

FIG. 1E depicts an exemplary ring portion for use in the exemplary prosthetic device shown in FIG. 1A;

FIG. 1F depicts a cross-sectional view of the exemplary ring portion along line A-A shown in FIG. 1E;

FIG. 1G depicts an exemplary cross-sectional view of another exemplary ring portion similar to the exemplary ring portion shown in FIG. 1E;

FIG. 2A depicts a view of another exemplary prosthetic device of the present invention;

FIG. 2B depicts exemplary inserts for use in the exemplary prosthetic device shown in FIG. 2A;

FIG. 2C depicts a single exemplary insert for use in the exemplary prosthetic device shown in FIG. 2A;

FIG. 3A depicts another exemplary prosthetic device of the present invention;

FIG. 3B depicts a cross-sectional view of the exemplary prosthetic device shown in FIG. 3A, as viewed along line B-B, showing features of an exemplary rigid member assembly suitable for use with any prosthetic device of the present invention;

FIG. 3C depicts a profile view of the exemplary rigid member shown in FIGS. 3A-3B;

FIG. 3D depicts a top view of an exemplary socket connector plate used in the exemplary prosthetic device shown in FIGS. 3A-3B;

FIG. 3E depicts a side view of an L-shaped member used in the exemplary prosthetic device shown in FIGS. 3A-3B;

FIG. 3F depicts a cross-sectional view of the foot portion of the exemplary prosthetic device shown in FIG. 3A, as viewed along line C-C, showing features of an exemplary foot suitable for use with any prosthetic device of the present invention;

FIG. 4 depicts a view of another exemplary prosthetic device of the present invention;

FIG. 4A depicts an exemplary knee joint suitable for use in any prosthetic device of the present invention;

FIG. 4B depicts the exemplary knee joint shown in FIG. 4A in a bent configuration;

FIG. 4C depicts a frontal view of the exemplary knee joint shown in FIG. 4A;

FIG. 4D depicts another exemplary knee joint suitable for use in any prosthetic device of the present invention;

FIG. 4E depicts a cross-sectional view of the exemplary knee joint shown in FIG. 4D as viewed along line D-D, showing an internal tensioning member;

FIG. 4F depicts a cross-sectional view of the exemplary knee joint shown in FIG. 4D as viewed along line D-D, in a bent configuration;

FIG. 4G depicts a frontal view of the exemplary knee joint shown in FIG. 4D;

FIG. 4H depicts a rear view of the exemplary knee joint shown in FIG. 4D; and

FIGS. 5A-5B depict optional rim features for any of the universal sockets used in the exemplary prosthetic devices of the present invention.

### DETAILED DESCRIPTION

To promote an understanding of the principles of the present invention, descriptions of specific embodiments of the invention follow and specific language is used to describe the specific embodiments. It will nevertheless be understood that no limitation of the scope of the invention is intended by the use of specific language. Alterations, further modifications, and such further applications of the principles of the present invention discussed are contemplated as would normally occur to one ordinarily skilled in the art to which the invention pertains.

The present invention is directed to prosthetic devices. The present invention is further directed to methods of making prosthetic devices. The present invention is even further directed to methods of using prosthetic devices.

### I. Prosthetic Devices

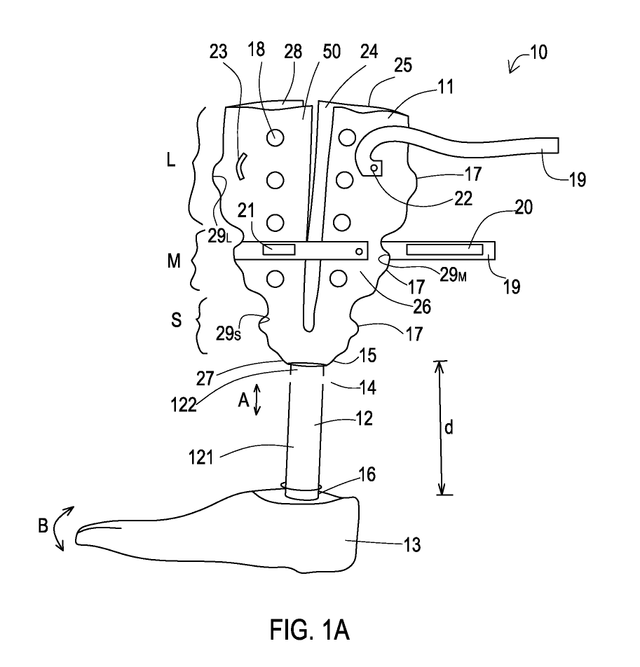

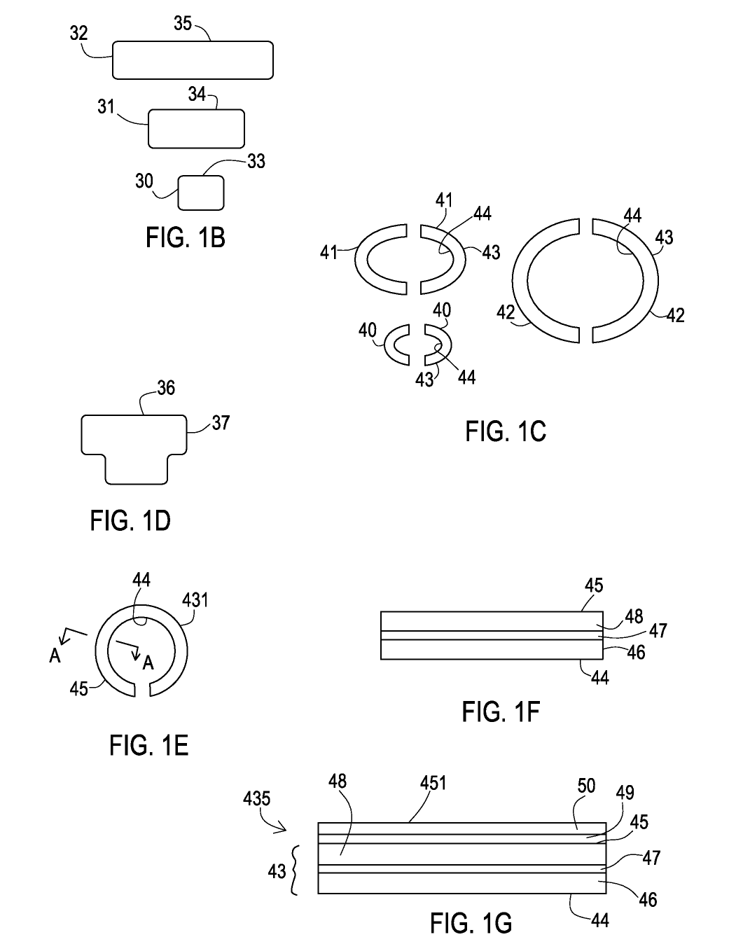

An exemplary prosthetic device of the present invention is shown in FIG. 1A. As shown in FIG. 1A, exemplary prosthetic device 10 comprises a universal socket 11 operatively adapted and sized to receive a variety of stump sizes (not shown). Universal socket 11 comprises a first socket open end 25 sized to receive a user stump (not shown), a second socket end 27 opposite the first socket end 25, at least two differently sized socket regions (shown as L, M and S) positioned between the first socket open end 25 and the second socket end 27. The at least two differently sized socket regions (shown as L and S) comprise an upper socket region (shown as L) proximate the first socket open end 25 and a lower socket region (shown as S) positioned between the upper socket region (shown as L) and the second socket end 27. The upper socket region (shown as L) has an upper region cross-sectional area; the lower socket region (shown as S) has a lower region cross-sectional area; and the upper region cross-sectional area is greater than the lower region cross-sectional area.

As shown in FIG. 1A, exemplary prosthetic device 10 comprises at least two differently sized socket regions, wherein the at least two differently sized socket regions comprise three differently sized socket regions comprising the upper socket region (shown as L), the lower socket region (shown as S), and a central socket region (shown as M) positioned between the upper socket region L and the lower socket region S, wherein the central socket region M has a central region cross-sectional area, and the central region cross-sectional area is less than the upper region cross-sectional area and greater than the lower region cross-sectional area.

Exemplary prosthetic device 10 may further comprise a universal socket 11, which further comprises concave grooves 17 along inner side wall surface 28 of universal socket 11 with at least one concave groove 17 in each of the at least two differently sized socket regions (shown as L and S).

Exemplary prosthetic device 10 may further comprise at least one slot 24 extending from the first socket open end 25 towards the second socket end 27. The at least one slot 24 may extend into the lower socket region (shown as S). Typically, the at least one slot 24 comprises two slots on opposite sides of the universal socket 11. However, it should be noted that in some embodiments, only one slot 24 is needed in a given exemplary prosthetic device.

Exemplary prosthetic device 10 may further comprise one or more openings 18 extending through a side wall 50 of the universal socket 11 from an outer surface 26 to an inner surface 28 of the universal socket 11. One or more openings 18 provide desired air flow through universal socket 11, as well as added traction between universal socket 11 and a user's stump (not shown).

Exemplary prosthetic device 10 may further comprise one or more tightening elements operatively adapted to tighten the universal socket 11 onto a user's stump positioned within the universal socket 11 (not shown). In one exemplary embodiment, and as shown in FIG. 1A, the one or more tightening elements comprise one or more straps 19 extending along an outer surface 26 of the universal socket 11. Each strap may desirably have (i) a length so as to be capable of extending along an outer perimeter of the universal socket 11, and (ii) at least one fastening component that enables one portion of the strap (i.e., 20) to attach to another portion of the strap (i.e., 21). For example, the at least one fastening component may comprise hook and loop material (i.e., 20, 21). Each strap 19 may be attached to an outer surface 26 of the universal socket 11 via a strap attachment member 22. The universal socket 11 may further comprise one or more strap loops 23 positioned along the outer surface 26 of universal socket 11 in order to control a position of a given strap 19 along the outer surface 26 of universal socket 11.

It should be noted that any tightening element(s) may be used to tighten the universal socket 11 onto a user's stump positioned within the universal socket 11 (not shown). Suitable tightening elements include, but are not limited to, male/female clasping devices positioned on either side of at least one slot 24 (e.g., clasping devices typically used on ski boots or roller blades); rubber or other elastomeric band materials that extend around a perimeter of universal socket 11; hook and loop material in combination with one or more clasps positioned on either side of at least one slot 24 (e.g., the hook and loop material does not extend completely around universal socket 11); or any combination of one or more types of tightening elements.

As shown in FIG. 1B, exemplary prosthetic device 10 may further comprise at least one stump cushion sized so as to be positionable within the universal socket 11. Exemplary stump cushions 30, 31 and 32 are shown in FIG. 1B. Each stump cushion (e.g., exemplary stump cushions 30, 31 and 32) is sized so as to have a cross-sectional area substantially similar to a cross-sectional area of at least one socket region (e.g., regions L, M and S) within the universal socket 11. In one exemplary embodiment, the at least one stump cushion comprises multiple stump cushions (e.g., exemplary stump cushions 30, 31 and 32) with at least one stump cushion sized to fit within each of the at least two differently sized socket regions (e.g., regions L, M and S).

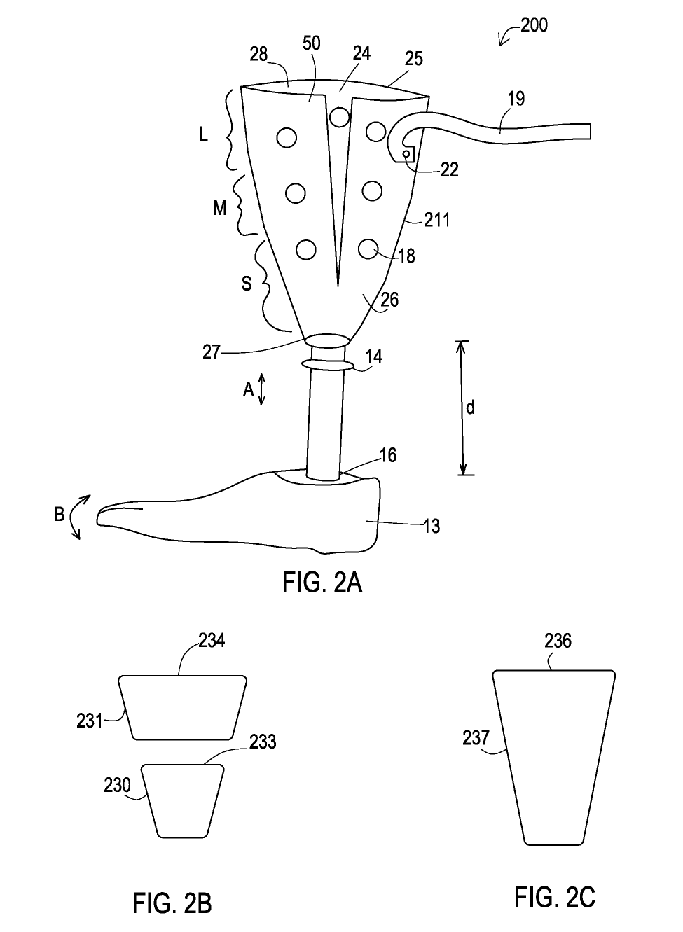

In some exemplary embodiments, the at least one stump cushion, when positioned in a socket region other than the lower socket region (e.g., other than region S), is positionable within the universal socket 11 so as to occupy substantially all of a volume of space bordered by an upper surface of the at least one stump cushion (e.g., upper surfaces 34 and 35 of exemplary stump cushions 31 and 32 respectively), inner side walls 28 of the universal socket 11, and a lower inner surface of the universal socket 11 proximate second socket end 27. See, for example, exemplary stump cushions 37 and 237 shown in FIGS. 1D and 2C respectively.

In other exemplary embodiments, the at least one stump cushion, when positioned in a socket region other than the lower socket region (e.g., other than region S), is positionable within the universal socket 11 so as to occupy less than a complete volume of space bordered by an upper surface of the at least one stump cushion (e.g., upper surfaces 34 and 35 of exemplary stump cushions 31 and 32 respectively), inner side walls 28 of the universal socket 11, and a lower inner surface of the universal socket 11.

Although exemplary stump cushions 30, 31 and 32 are shown as having a particular shape, each of exemplary stump cushions 30, 31 and 32 may have any desired shape as long as exemplary stump cushions 30, 31 and 32 are positionable within the universal socket 11. For example, a single stump cushion may have a shape that provides an upper surface for contact with a user's stump, fits snugly within the universal socket 11, and fills a volume of the universal socket 11 below the upper surface of the single stump cushion. Such an exemplary single stump cushion is shown in FIG. 1D. As shown in FIG. 1D, exemplary single stump cushion 37 comprises upper surface 36, and a cushion size and shape so as to occupy a complete volume of space bordered by upper surface 36, inner side walls 28 of the universal socket 11, and a lower inner surface of the universal socket 11 when placed within universal socket 11.

Suitable materials for forming any of the disclosed stump cushions, including exemplary stump cushions 30, 31, 32 and 37, include, but are not limited to, a silicone or foam material (e.g., a high density foam material).

As shown in FIG. 1C, exemplary prosthetic device 10 may further comprise at least one pair of ring sections (e.g., exemplary ring sections 40, 41 and 42), wherein each pair of ring sections (i) has an outer ring surface 43 operatively adapted to contact an inner surface 28 of the universal socket 11 in a non-slip manner, (ii) an inner ring surface 44 operatively adapted to contact an outer surface of a user's stump (not shown) when positioned within the universal socket 11, and (iii) is sized so as to extend along an inner perimeter surface 28 of the universal socket 11. In one exemplary embodiment, the at least one pair of ring sections comprises at least one pair of ring sections for each of the at least two differently sized socket regions (e.g., exemplary ring section 40 for socket region S and longer exemplary ring section 42 for socket region L). In other exemplary embodiments, the at least one pair of ring sections comprises at least one pair of ring sections (e.g., exemplary ring sections 40 and 42) configured to extend along an inner perimeter surface (e.g., depicted as inner perimeter surfaces 29_{L}, 29_{M} and 29_{S}) of concave grooves 17 along inner side walls 28 of a concave groove 17 in each of the at least two differently sized socket regions (i.e., along inner perimeter surfaces 29_{L}, 29_{M} and 29_{S} of concave grooves 17 in each of regions L, M and S). In one desired embodiment, the outer ring surface 43 of a given pair of ring sections is operatively adapted to contact and adhere to the inner surface 28 of the universal socket 11. Further, desirably, the at least one pair of ring sections (e.g., exemplary ring sections 40, 41 and 42) is operatively adapted to accept a partial load from a user's stump (not shown) when positioned within the universal socket 11.

It should be noted that in some embodiments of the present invention, a single ring section may be used in place of a pair of ring sections. For example, an exemplary single ring section 431 is shown in FIG. 1E. Exemplary single ring section 431 may be sized so as to extend substantially along a complete perimeter length of inner surface 28 of the universal socket 11 (i.e., substantially along inner perimeter surfaces 29_{L}, 29_{M} and 29_{S} of concave grooves 17 in each of regions L, M and S especially in embodiments wherein universal socket 11 comprises a single slot 24 therein).

Suitable materials for forming exemplary ring sections 40, 41, 42 and 431 include, but are not limited to, a silicone or foam material (e.g., a low density foam material). Various cross-sectional configurations for exemplary single ring section 431 (which are also applicable for exemplary ring section 40, 41 and 42) are depicted in FIGS. 1F-1G.

FIG. 1F depicts a cross-sectional view of exemplary ring portion 431 along line A-A shown in FIG. 1E. As shown in FIG. 1F, exemplary ring portion 431 comprises a first layer 46 having outer surface 44, which contacts a user's stump or clothing positioned over a user's stump (not shown); a second layer 48 having outer surface 45, which contacts, directly or indirectly, inner surface 28 of universal socket 11; and a third layer 47 positioned between first layer 46 and second layer 48. Third layer 47 may comprise any type of adhesive suitable for bonding first layer 46 to second layer 48 (e.g., a pressure sensitive adhesive (PSA), a heat activatable adhesive, etc.).

In the exemplary embodiment shown in FIG. 1F, outer surface 45 may have a degree of tack that enables exemplary ring portion 431 to at least temporarily adhere to inner surface 28 of universal socket 11. Otherwise, an adhesive material may be applied onto at least a portion of outer surface 45 so as to provide a degree of adhesive to enable exemplary ring portion 431 to at least temporarily adhere to inner surface 28 of universal socket 11. Any type of adhesive may be used to bond outer surface 45 to inner surface 28 of universal socket 11 (e.g., a pressure sensitive adhesive (PSA), a heat activatable adhesive, etc.).

Typically, first layer 46 having outer surface 44 comprises a relatively soft, silicone rubber material, while second layer 48 having outer surface 45 comprises a relatively harder material, such as a high density foam material (e.g., any relatively inexpensive foam material). Suitable silicone rubber materials and high density foam materials are commercially available from American Plastics (Arlington, Tex.). One suitable high density foam material commercially available from American Plastics is sold under the TRI-LAM™ trade designation.

FIG. 1G depicts an exemplary cross-sectional view of another exemplary ring portion 435 similar to exemplary ring portion 431 shown in FIG. 1E. As shown in FIG. 1G, exemplary ring portion 435 comprises first layer 46; a second layer 48; and a third layer 47 positioned between first layer 46 and second layer 48 as discussed above. In addition, exemplary ring portion 435 comprises an adhesive layer 49 positioned over at least a portion, desirably all, of outer surface 45. Desirably, adhesive layer 49 comprises a pressure sensitive adhesive (PSA) that adheres to inner surface 28 of universal socket 11 upon contact. As shown in FIG. 1G, exemplary ring portion 435 further comprises a removable release liner 50 positioned over and covering adhesive layer 49.

In the exemplary embodiment of FIG. 1G, a user simply removes release liner 50 and applies pressure onto exemplary ring portion 435 so as to bond outer adhesive surface 451 of adhesive layer 49 to inner surface 28 of universal socket 11 (e.g., along inner perimeter surfaces 29_{L}, 29_{M} and 29_{S} of concave grooves 17 in each of regions L, M and S within universal socket 11).

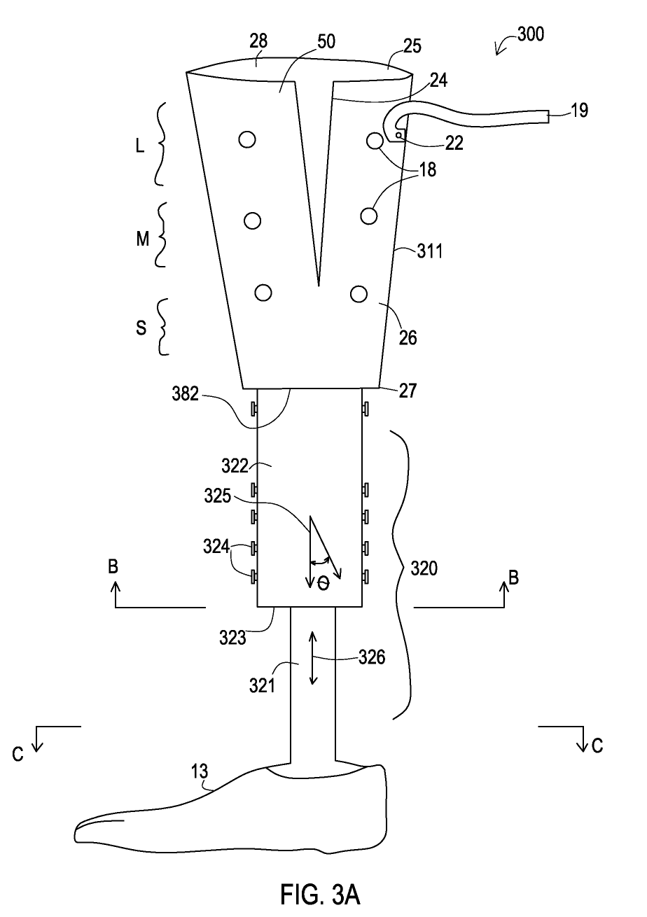

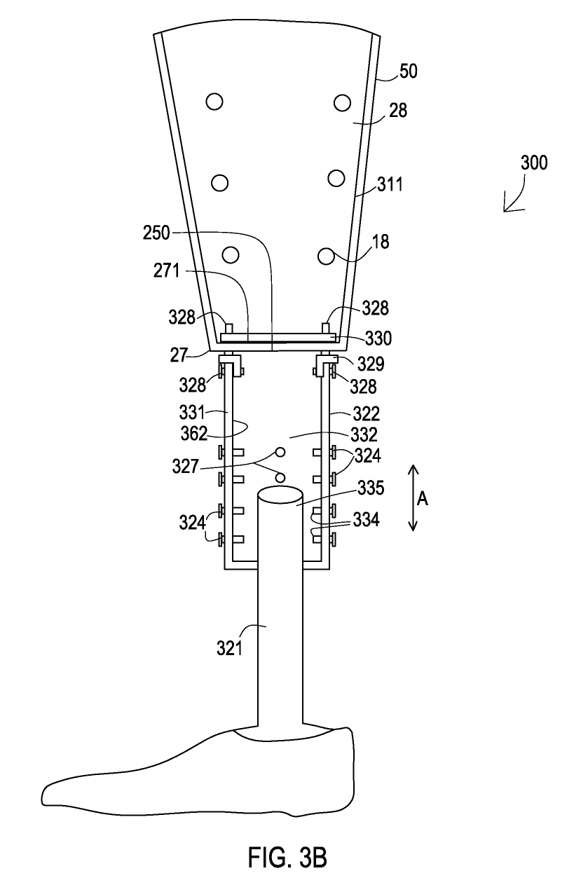

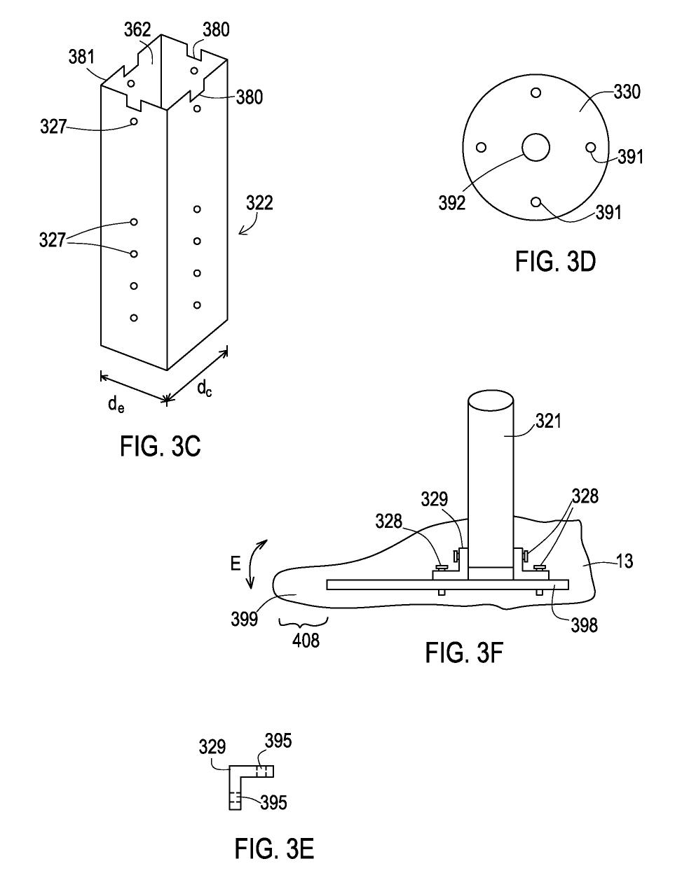

As shown in FIG. 1A, exemplary prosthetic device 10 may further comprise an artificial foot member 13; and a rigid member 12 extending from and connecting the second socket end 27 via a socket connector 15 to the artificial foot member 13. Exemplary prosthetic device 10 may further comprise a foot connector 16 positioned between a lower end of the rigid member 12 and the artificial foot member 13 so as to (i) limit movement of artificial foot member 13 relative to rigid member 12, and/or (ii) enable artificial foot member 16 to move in one or more directions relative to rigid member 12 (e.g., in a direction as shown by arrow B). In other exemplary embodiments (discussed below), rigid member 12 extends into artificial foot member 13 (see, FIG. 3F). Typically, artificial foot member 13 is stationary relative to rigid member 12.

Desirably, rigid member 12 is operatively adapted to (i) change in length so that a distance d between second socket end 27 and artificial foot member 13 is adjustable, and (ii) lock into a position so as to provide a fixed distance between second socket end 27 and artificial foot member 13. Any length adjustment/locking mechanism 14 may be used to accomplish these features. For example, in one exemplary embodiment, rigid member 12 comprises two tubular members 121 and 122 (e.g., aluminum tubular members), wherein upper tubular member 122 has a larger inner diameter to allow lower tubular member 121 to slide within upper tubular member 122. Once lower tubular member 121 is positioned in a desired location within upper tubular member 122, length adjustment/locking mechanism 14 may be used to securely fix a position of lower tubular member 121 relative to upper tubular member 122.

Suitable length adjustment/locking mechanisms 14 include, but are not limited to, a clamp positioned over portions of lower tubular member 121 and upper tubular member 122; a coupling capable of attaching to an outer surface (e.g., threads along the outer surface) of each of lower tubular member 121 and upper tubular member 122; male/female coupling members positioned along lower tubular member 121 and upper tubular member 122 (e.g., grooves within upper tubular member 122 and an engaging member for engaging with one or more grooves along lower tubular member 121; and fastening members (e.g., screws) extending from an outer upper tubular member 122 toward in inner lower tubular member 121 for securing lower tubular member 121 in a position relative to upper tubular member 122 (i.e., see discussion below regarding upper rigid member 322 and lower rigid member 321 shown in FIGS. 3A-3F).

The above-described exemplary prosthetic device 10 shown in FIG. 1A represents a class of prosthetic devices of the present invention, wherein the universal sockets having a “non-tapered configuration.” As shown in exemplary prosthetic device 10, at least two differently sized socket regions L, M and S each comprise socket regions, wherein each socket region has a side wall component that extends substantially vertically, and each side wall component of a given socket region is separated from an adjacent socket region by a side wall component that extends both horizontally, and typically both horizontally and vertically. In order words, a given socket region, such as socket region L, has a cross-sectional area that remains relatively constant along at least a portion of a side wall extending along socket region L (i.e., the cross-sectional area is not continuously increasing as in a tapered configuration).

Other prosthetic devices of the present invention comprise universal sockets having a “tapered configuration” as shown in FIG. 2A. As shown in FIG. 2A, exemplary prosthetic device 200 has a tapered configuration, wherein the at least two differently sized socket regions L, M and S comprise socket regions, wherein each socket region has a side wall component that appears as an extension of the side wall of an adjacent socket region. In other words, the side wall of the universal socket 211 gradually increases from a smaller cross-sectional area proximate second socket end 27 to a largest cross-sectional area proximate first, open socket end 25.

As shown in FIG. 2A, exemplary prosthetic device 200 comprises many of the above-described features found in exemplary prosthetic device 10 including, but not limited to, first socket open end 25 sized to receive a user stump (not shown); a second socket end 27 opposite the first socket end 25; at least two differently sized socket regions (shown as L, M and S) positioned between first socket open end 25 and second socket end 27; inner side wall surface 28 of universal socket 211; at least one slot 24 (shown here as a single slot 24); one or more openings 18 extending through side wall 50 of universal socket 211 from an outer wall surface 26 to inner wall surface 28; and one or more tightening elements (e.g., one or more straps 19 attached to outer wall surface 26 via a strap attachment member 22).

As shown in FIGS. 2B-2C, at least one stump cushion specifically sized to be positionable within universal socket 211 may be used in combination with universal socket 211 as discussed above. Exemplary stump cushions 230 and 231 are shown in FIG. 2B. Exemplary stump cushions 230 and 231 are sized so as to have a cross-sectional area substantially similar to a cross-sectional area of at least one socket region (e.g., regions L and S) within universal socket 211. As discussed above, in some exemplary embodiments, the at least one stump cushion comprises an exemplary stump cushion sized to fit within each of the at least two differently sized socket regions (e.g., regions L, M and S).

As shown in FIG. 2C, exemplary single stump cushion 237 comprises upper surface 236, and a cushion size and shape so as to occupy a complete volume of space bordered by upper surface 36, inner side walls 28 of universal socket 211 and a lower inner surface of the universal socket 211 when placed within universal socket 211. Exemplary single stump cushion 237 may be used instead of two or more stump cushions (e.g., exemplary stump cushions 230 and 231 are shown in FIG. 2B).

Suitable materials for forming any of the disclosed stump cushions, including exemplary stump cushions 230, 231 and 237, include, but are not limited to, a silicone or foam material (e.g., a high density foam material) as discussed above.

FIG. 3A depicts yet another exemplary prosthetic device of the present invention. As shown in FIG. 3A, exemplary prosthetic device 300 comprises many of the above-described features found in exemplary prosthetic devices 10 and 200 including, but not limited to, first socket open end 25 sized to receive a user stump (not shown); a second socket end 27 opposite the first socket end 25; at least two differently sized socket regions (shown as L, M and S) positioned between first socket open end 25 and second socket end 27; inner side wall surface 28 of universal socket 311; at least one slot 24 (shown here as a single slot 24); one or more openings 18 extending through side wall 50 of universal socket 311 from an outer wall surface 26 to inner wall surface 28; and one or more tightening elements (e.g., one or more straps 19 attached to outer wall surface 26 via a strap attachment member 22).

Exemplary prosthetic device 300 further comprises a rigid member assembly 320 comprising upper rigid member 322 (e.g., an aluminum tubular member) and lower rigid member 321 (e.g., an aluminum solid or tubular member). Lower rigid member 321 is sized so as to be insertable within a lower end 323 of upper rigid member 322. Further, lower rigid member 321 is attached to upper rigid member 322 via one or more fasteners (e.g., screws) 324 so as to connect to upper rigid member 322 and orient lower rigid member 321 relative to a dissecting line 325 extending through and dissecting upper rigid member 322 along a centrally located axis. In some embodiments, lower rigid member 321 is attached to upper rigid member 322 so that a dissecting line 326 extending through and dissecting lower rigid member 321 (along along a centrally located axis of lower rigid member 321) is substantially in alignment with dissecting line 325 (as shown in exemplary prosthetic device 300 in FIG. 3A). In other embodiments, lower rigid member 321 is attached to upper rigid member 322 so that dissecting line 326 of lower rigid member 321 forms an angle θ with dissecting line 325. Typically, angle θ ranges from 0° to about 25°, more typically, from 0° to about 15°.

Each of upper rigid member 322 and lower rigid member 321 may independently have any length, width, and cross-sectional configuration so as to enable (i) an adjustable length between second socket end 27 and artificial foot member 13, (ii) connection of lower rigid member 321 to upper rigid member 322, and (iii) a desired angle θ. In one exemplary embodiment, upper rigid member 322 comprises a tubular member having a square cross-sectional configuration, while lower rigid member 321 comprises a solid (i.e., not hollow) or tubular member having a circular cross-sectional configuration.

As shown in FIG. 3A, rigid member assembly 320 comprises rows of fasteners 324 along opposite sides of upper rigid member 322. Desirably, a given rigid member assembly 320 comprises at least two rows of first fasteners 324 along an outer perimeter (at least a lower outer perimeter) of and encircling upper rigid member 322; however, in some embodiments, rigid member assembly 320 may comprise any number of rows of first fasteners 324 along an outer perimeter of and encircling upper rigid member 322 although a typical number of rows of first fasteners 324 ranges from about 2 to about 10 rows.

FIG. 3B depicts a cross-sectional view of exemplary prosthetic device 300. As shown in FIG. 3B, first fasteners (e.g., screws) 324 extend through holes 327 positioned within at least one side wall 331 of upper rigid member 322 into an interior volume 332 of upper rigid member 322. Although not shown, ends 334 of first fasteners (e.g., screws) 324 contact an outer surface 335 of lower rigid member 321 (or extend into an indentation or hole (not shown) within outer surface 335 of lower rigid member 321) so as to secure lower rigid member 321 relative to upper rigid member 322. Prior to being secured in a particular position, lower rigid member 321 may be moved in a direction, as shown by arrow A, so as to adjust an overall length of the combined upper rigid member 322 and lower rigid member 321.

FIG. 3B also shows one exemplary method of connecting upper rigid member 322 to second socket end 27 of universal socket 311. As shown in FIG. 3B, upper rigid member 322 is attached to universal socket 311 via second fasteners (e.g., screws) 328, L-shaped members 329, and a socket connector plate 330. In this exemplary embodiment, a first set of second fasteners (e.g., screws) 328 attaches a set of L-shaped members 329 to upper rigid member 322, desirably, an inner surface 362 of upper rigid member 322, while a second set of second fasteners (e.g., screws) 328 attaches the set of L-shaped members 329 to socket connector plate 330 positioned within and along (or proximate) a lower surface 271 of universal socket 311. Each second fastener within the second set of second fasteners (e.g., screws) 328 extends through (i) holes 391 within socket connector plate 330 and (ii) a lower wall 250 of universal socket 311 in one or more locations.

FIG. 3C depicts a profile view of exemplary upper rigid member 322. As shown in FIG. 3C, exemplary upper rigid member 322 has a four-sided cross-sectional configuration with side dimensions dₑ and dₑ. Desirably, side dimensions dₑ and dₑ are equal so as to provide a square cross-sectional configuration. Desirably, exemplary upper rigid member 322 further comprises cut-out sections 380 within upper edge 381 so as to accommodate a cross-sectional portion of a given L-shaped member 329 (not shown; see, FIG. 3B) positioned therein so that upper edge 381 of upper rigid member 322 rests along a lower surface 382 of universal socket 311.

FIG. 3D depicts a top view of exemplary socket connector plate 330 showing holes 391 through which second set of second fasteners (e.g., screws) 328 extend. Socket connector plate 330 may further comprise an optional central opening 392 as shown in FIG. 3D.

FIG. 3E depicts a side view of an exemplary L-shaped member 329 used in exemplary prosthetic device 300 shown in FIGS. 3A-3B. Dashed lines are used to indicate locations of holes 395 within exemplary L-shaped member 329.

FIG. 3F depicts a cross-sectional view of exemplary foot 13 of exemplary prosthetic device 300 shown in FIG. 3A, as viewed along line C-C, showing features of an exemplary foot 13 that are suitable for use with any prosthetic device of the present invention. As shown in FIG. 3F, exemplary foot 13 may be connected to lower rigid member 321 via one or more L-shaped members 329, one or more second fasteners (e.g., screws) 328, and a foot plate 398 embedded within a foot material 399. Foot plate 398 may comprise, for example, an aluminum plate having a plate thickness of about 1.5 millimeters (mm) Desirably, foot plate 398 extends a length along foot 13 so as to provide structural integrity within foot 13 while allowing a toe portion 408 of foot 13 to move in a direction, as shown by arrow E, when exposed to an external force (e.g., when the toe portion 408 of foot 13 is pushing off the ground during walking).

Although not shown in combination with second fasteners (e.g., screws) 328, it should be understood that one or more bolts may be used in combination with second fasteners (e.g., screws) 328 to secure any of the above-mentioned components to one another. Further, although not shown in FIGS. 3A-3F, it should be understood that any of the above-mentioned components (e.g., exemplary stump cushions 30, 31, 32, 230 and 231; exemplary single stump cushions 37 and 237; exemplary ring sections 40, 41 and 42; and exemplary single ring section 431) may be used in combination with exemplary prosthetic device 300.

It should be further noted that there is no limitation on the materials used to form the components described herein. For example, any of the above-described universal sockets may be formed from any thermoformable material, desirably a relatively inexpensive material such as polyethylene or polypropylene. Any of the rigid members may be formed from metallic, polymeric or composite materials (e.g., fiber-reinforced polymeric material), although aluminum is a preferred material. Further, the artificial foot may be formed by any thermoformable material, but is desirably a material such as CREPE neoprene material.

Exemplary prosthetic device 10 shown in FIG. 1A, exemplary prosthetic device 200 shown in FIG. 2A, and exemplary prosthetic device 300 shown in FIG. 3A are referred to herein as “below-the-knee” prosthetic devices of the present invention. These “below-the-knee” prosthetic devices are specifically sized and designed for use with amputees (i.e., from small children to large adults) having a stump below the knee. In other embodiments of the present invention, described below, the prosthetic devices further comprise a knee joint, and are referred to herein as “above-the-knee” prosthetic devices of the present invention.

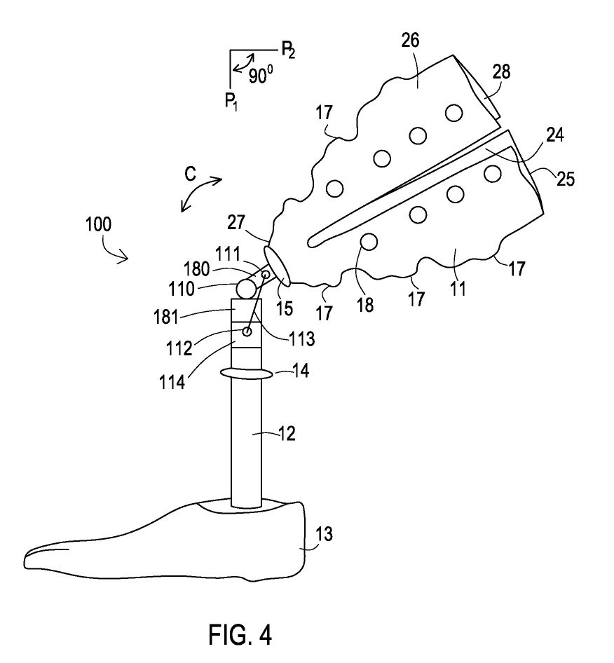

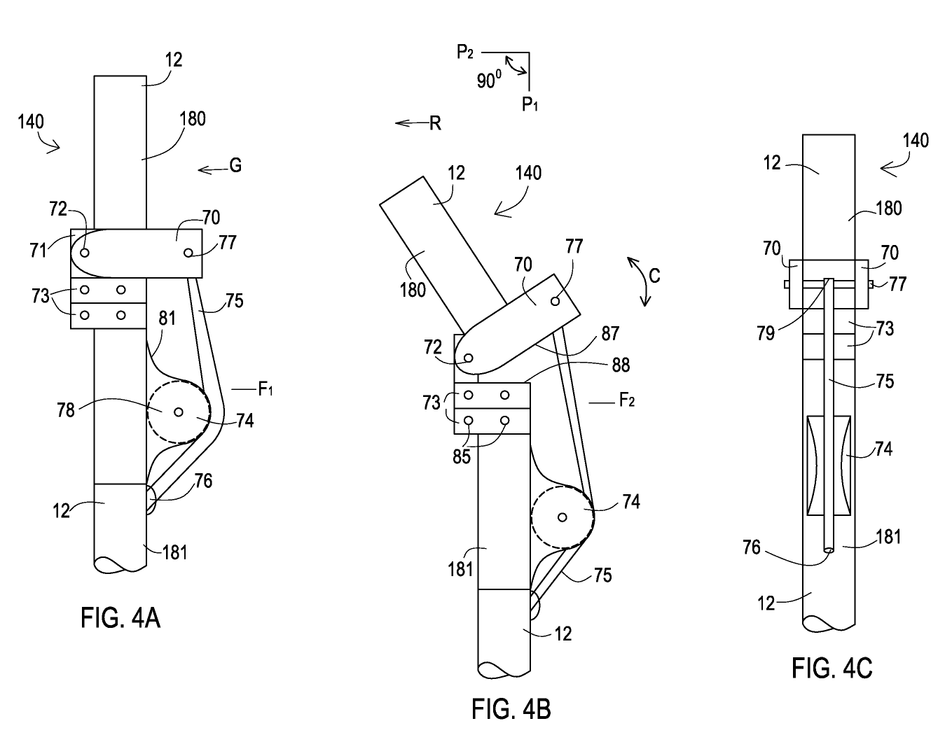

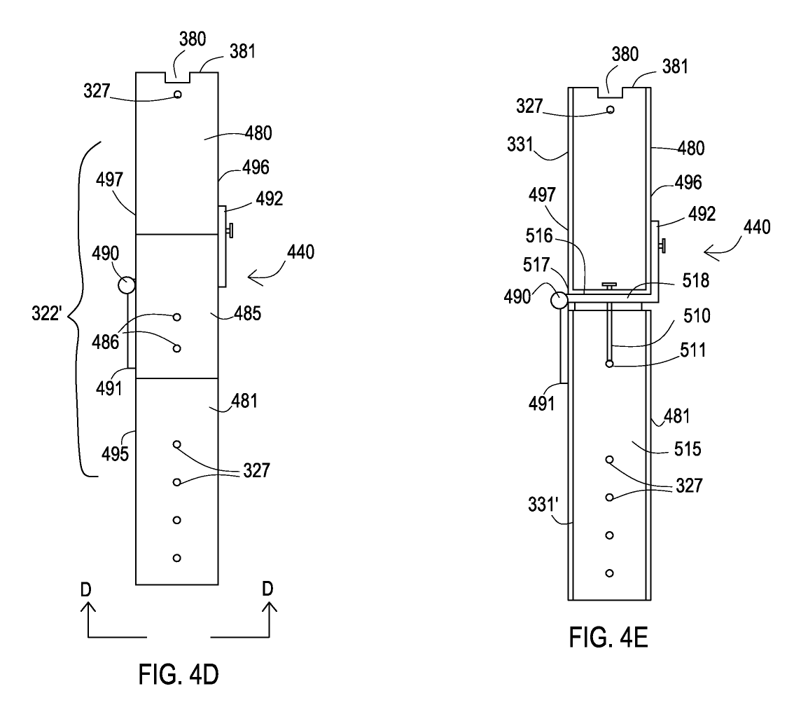

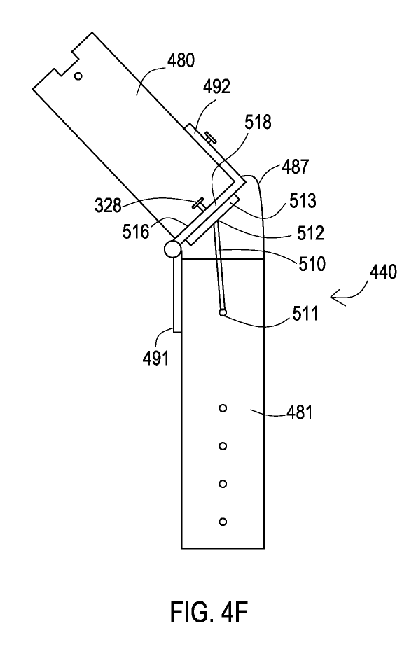

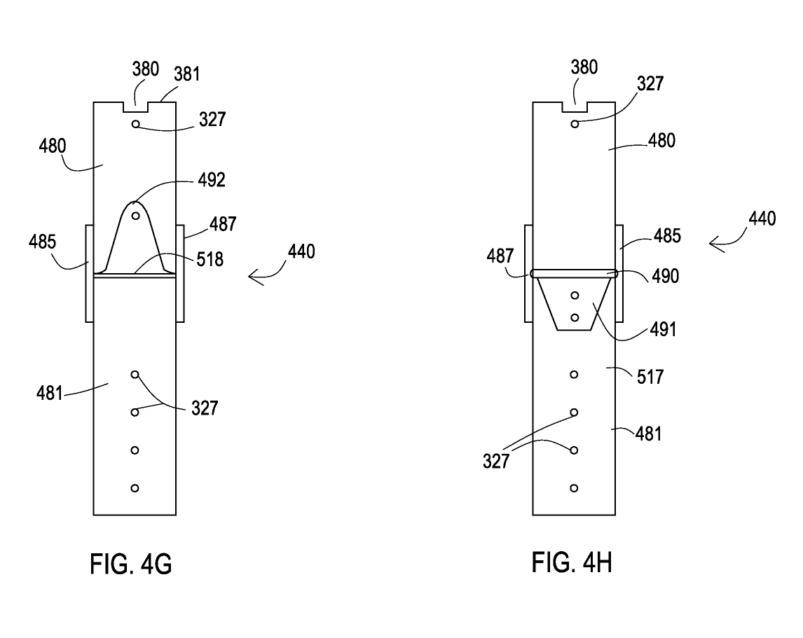

In “above-the-knee” prosthetic devices of the present invention, rigid member 12 (or upper rigid member 322) further comprises an artificial knee joint positioned along rigid member 12 (or upper rigid member 322). Such an exemplary embodiment is shown in FIG. 4. As shown in FIG. 4, exemplary prosthetic device 100 comprises many of the above-mentioned features, as well as artificial knee joint 110 positioned along rigid member 12. Artificial knee joint 110 enables controlled bending of rigid member 12 at artificial knee joint 110 in a direction as shown by arrow C. Typically, artificial knee joint 110 enables controlled bending of rigid member 12 of about 90° in a direction as shown by arrow C and as shown by position P₁ and position P₂.

Typically, rigid member 12 is configured to remain in a straight configuration (i.e., in position P₁) at artificial knee joint 110, and a desired amount of tension is necessary to move rigid member 12 into an angled configuration (i.e., in position P₂). Any tensioning system may be used to apply a desired amount of tension at artificial knee joint 110. In exemplary prosthetic device 100 shown in FIG. 3, artificial knee joint 110 comprises a spring-loaded joint that keeps rigid member 12 in a straight configuration (i.e., in position P₁) at artificial knee joint 110 when in a relaxed state. Cables 113 extends on either side of artificial knee joint 110 and attach an upper section 180 of rigid member 12 (at location 111) to a lower section 181 of rigid member 12 (at location 112).

FIGS. 4A-4C depict various views of an alternative exemplary knee joint suitable for use in any of the herein described prosthetic devices. As shown in FIG. 4A, exemplary knee joint 140 comprises upper section 180 and lower section 181 of rigid member 12. Upper section 180 has attached thereto a movable tensioning member 70. Movable tensioning member 70 is pivotably mounted at pivot point 72 (i.e., bolt 72) to vertically extending member 71, which is connected to horizontally extending members 73 (via bolts 85). Horizontally extending members 73 are attached to lower section 181 of rigid member 12 (via bolts 85). A tensioning cable 75 is attached to movable tensioning member 70 along bolt 77, extends from movable tensioning member 70, over cable guide 74 positioned along an outer surface 81 of lower section 181 of rigid member 12, and is attached to outer surface 81 of lower section 181 of rigid member 12 at location 76. Cable guide 74 may simply comprise a smooth surface or may alternatively comprise a wheel 78 mounted along axis 82 of cable guide 74.

Tensioning cable 75 may be any elastomeric cable material. Suitable materials for forming tensioning cable 75 include, but are not limited to, a bungee cord type material, a spring, any elastic material, or a combination thereof.

FIG. 4A depicts exemplary knee joint 140 in a relaxed state, wherein the tension force along tensioning cable 75 is F₁. As shown in FIG. 4B, exemplary knee joint 140 may be moved in a direction as shown by arrow R so as to move exemplary knee joint 140 into an angled configuration. As increased force is applies onto upper section 180 in direction R, movable tensioning member 70 pivots at pivot point 72 (i.e., bolt 72) so as to separate a lower surface 87 of movable tensioning member 70 from an upper surface 88 of upper horizontally extending member 73 in a direction as shown by arrow C. As force is decreased on upper section 180, movable tensioning member 70 pivots at pivot point 72 (i.e., bolt 72) so as to return to a relaxed position as shown in FIG. 4A.

FIG. 4B depicts exemplary knee joint 140 in a non-relaxed (i.e., increased tension) state, wherein the tension force along tensioning cable 75 is F₂, and F₂ is greater than F₁.

FIG. 4C depicts a frontal view of exemplary knee joint 140 as viewed along arrow G shown in FIG. 4A. As shown in FIG. 4C, tensioning cable 75 is attached to bolt 77 at location 79, extends from movable tensioning member 70, over cable guide 74 positioned along outer surface 81 of lower section 181, and is attached to outer surface 81 of lower section 181 of rigid member 12 at location 76. Tensioning cable 75 may be attached to bolt 77 and location 76 via any known attachment member. For example, tensioning member 70 may comprise hooks on either end so as to attach to bolt 77 and through a loop (not shown) fixed along outer surface 81 of lower section 181 at location 76.

Although not shown in FIGS. 4A-4C, upper section 180 is attachable to any of the above-described universal sockets of the present invention, while lower section 181 is attachable to an artificial foot (e.g., artificial foot member 13). Further, although not shown in FIGS. 4A-4C, each of upper section 180 and lower section 181 may independently comprise a length adjustment/locking mechanism, such as exemplary length adjustment/locking mechanism 14 described above, so as to adjust and securely fix a length of each of upper section 180 and lower section 181.

FIGS. 4D-4H depict various views of an alternative exemplary artificial knee joint 440 suitable for use in any of the prosthetic devices of the present invention. As shown in FIG. 4D, exemplary knee joint 440 comprises upper rigid member section 480 and lower rigid member section 481 of rigid member 322′. Upper rigid member section 480 comprises cut-out sections 380 within upper edge 381 so as to accommodate a cross-sectional portion of a given L-shaped member 329 (not shown) positioned therein so that upper edge 381 of upper rigid member 322′ rests along a lower surface of one of the universal sockets disclosed herein (e.g., along a lower surface 382 of universal socket 311 as shown in FIG. 3A). Upper rigid member section 480 also comprises holes 327 positioned within at least one side wall 331 (see FIG. 4E) of upper rigid member section 480 so as to connect upper rigid member section 480 to a universal socket via, for example, via second fasteners (e.g., screws) 328, L-shaped members 329, and a socket connector plate 330 as described above.

Lower rigid member section 481 of rigid member 322′ comprises rows of holes 327 positioned within at least one side wall 331′ to accommodate first fasteners (e.g., screws) (e.g., first fasteners 324) for contacting and securing a lower rigid member (not shown), such as lower rigid member 321, within lower rigid member section 481 of rigid member 322′. Lower rigid member section 481 further comprises an optional first guide member 485 attached to an upper region thereof via fasteners (e.g., screws) 486. As discussed further below, first guide member 485 and second guide member 487 (shown in FIGS. 4F-4H) provide guidance for restricted lateral movement of upper rigid member section 480 when upper rigid member section 480 moves relative to lower rigid member section 481.

Exemplary knee joint 440 further comprises a hinge member 490 having a first hinge end 491 attached to lower rigid member section 481 and a second hinge end 492 attached to upper rigid member section 480. It should be noted that although exemplary hinge member 490 is shown in a desired configuration with first hinge end 491 attached to a rear surface 495 of lower rigid member section 481 and second hinge end 492 attached to a front surface 496 of upper rigid member section 480, exemplary hinge member 490 could be configured so that first hinge end 491 is attached to rear surface 495 of lower rigid member section 481 and second hinge end 492 is attached to a rear surface 497 of upper rigid member section 480.

FIG. 4E depicts a cross-sectional view of exemplary knee joint 440 shown in FIG. 4D as viewed along line D-D, when exemplary knee joint 440 is in a “relaxed” state. As shown in FIG. 4E, exemplary knee joint 440 further comprises a tensioning member 510 positioned within lower rigid member section 481. In this exemplary embodiment, tensioning member 510 connects to a plate 513 and a stationary member 511 positioned within lower rigid member section 481. Stationary member 511 may be, for example, a screw, bolt or rod extending from an interior surface 515 of lower rigid member section 481 or between opposite interior surfaces 515 within lower rigid member section 481.

As shown in FIG. 4E, a portion 518 of exemplary hinge member 490 extends from a rear side 517 of upper rigid member section 480 to front surface 496 along a lower edge 516 of upper rigid member section 380. Portion 518 of exemplary hinge member 490 provides a surface onto which tensioning member 510 may be attached or plate 513 may be attached. Tensioning member 510 may be attached directly to portion 518 of exemplary hinge member 490 or indirectly to portion 518 of exemplary hinge member 490 via plate 513. Any attachment member may be used to attach tensioning member 510 to portion 518 of exemplary hinge member 490 or plate 513 including, but not limited to, a screw, a bolt or an indentation within exemplary hinge member 490 or plate 513. In this exemplary embodiment, tensioning member 510 is attached to plate 513, which is attached to portion 518 of exemplary hinge member 490 via one or more fasteners (e.g., screws).

FIG. 4F depicts a cross-sectional view of exemplary knee joint 440 shown in FIG. 4D as viewed along line D-D, when exemplary knee joint 440 is in a bent, tensioned (i.e., a non-relaxed) configuration. As shown in FIG. 4F, tensioning member 510 has a tension force therein that is greater than the tension force within tensioning member 510 as shown in FIG. 4E. Tensioning member 510 provides a tension force between upper rigid member section 480 and lower rigid member section 481 so as to return exemplary knee joint 440 to a relaxed configuration as shown in FIG. 4E when an external force is removed from exemplary knee joint 440.

FIG. 4G depicts a frontal view of exemplary knee joint 440 shown in FIG. 4D while FIG. 4H depicts a rear view of exemplary knee joint 440 shown in FIG. 4D. As shown in FIGS. 4G-4H, first guide member 485 and second guide member 487 are attached to opposite sides of lower rigid member section 481 so as to restrict lateral movement of upper rigid member section 480 when upper rigid member section 480 moves relative to lower rigid member section 481 as shown in FIG. 4F. Although not shown, it should be noted that in an alternative embodiment, first guide member 485 and second guide member 487 could be attached to opposite sides of upper rigid member section 480 so as to restrict lateral movement of upper rigid member section 480 when upper rigid member section 480 moves relative to lower rigid member section 481 as shown in FIG. 4F.

Tensioning member 510 may be any tensioning material. Suitable tensioning materials for forming tensioning member 510 include, but are not limited to, a spring, a bungee cord type material, any other elastic material, or a combination thereof.

Although not shown in FIGS. 4D-4H, upper rigid member section 480 is attachable to any of the above-described universal sockets of the present invention, while lower rigid member section 481 is attachable to a lower rigid member (e.g., lower rigid member 321) and an artificial foot (e.g., artificial foot member 13). Further, although not shown in FIGS. 4D-4H, each of upper rigid member section 480 and lower rigid member section 481 and a corresponding lower rigid member (e.g., lower rigid member 321) may independently have any length, width, and cross-sectional configuration so as to enable (i) an adjustable length between second socket end 27 and artificial foot member 13, (ii) connection of a corresponding lower rigid member (e.g., lower rigid member 321) to lower rigid member section 481 of upper rigid member 322′, and (iii) a desired angle θ as discussed above.

The above-described exemplary prosthetic devices may be used immediately after an amputation operation to protect against injury during both early and preparatory stages of wound healing and the rehabilitation process. The above-described exemplary prosthetic devices provide access to bandages and dressings for wound care. Because of their ability to change and adjust the socket volume, the above-described exemplary prosthetic devices can accommodate compression and swelling of the distal stump's wound area, and accepts elastic wrap bandages to reduce swelling. Because the above-described exemplary prosthetic devices significantly reduce load on the distal stump, pressure sores and ulcers are less likely to develop at the distal end of the stump. Further, the above-described exemplary prosthetic devices allow ventilation via multiple holes 18, which may be randomly and/or evenly distributed along the socket wall to enhance postoperative healing. The above-described exemplary prosthetic devices also help maintain correct alignment in three planes of motion (i.e., frontal, saggital, and transverse planes) and shape and prepare the residual limb for a more permanent prosthesis if so desired.

The above-described exemplary prosthetic devices are easy to fit onto the amputee's stump without requiring any tools or laboratory set up.

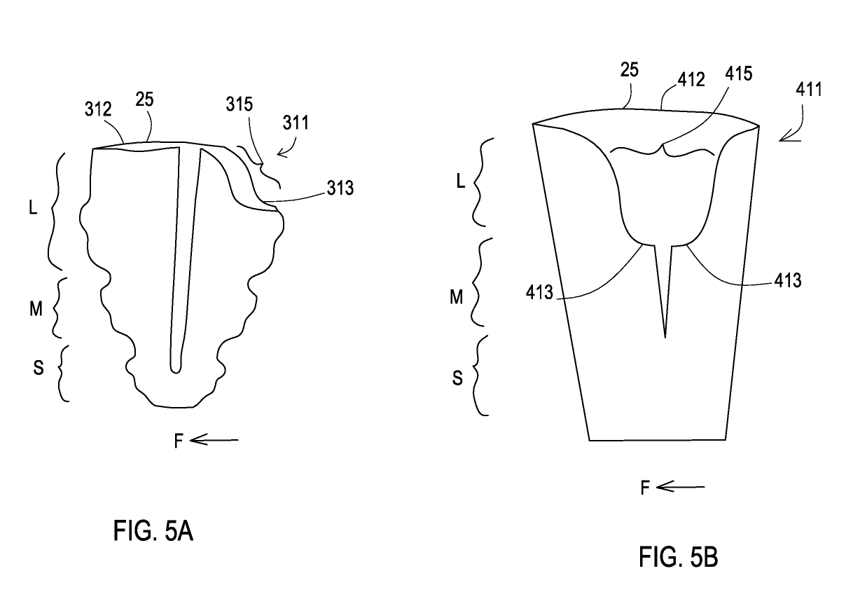

Any of the above-described exemplary prosthetic devices may further comprise features as shown in FIGS. 5A-5B. As shown in FIG. 5A, exemplary universal socket 311 may comprise first socket open end 25, wherein first socket open end 25 has a front rim section 312 that is at a higher position relative to a rear rim section 313. In FIG. 5A, arrow F is used to show a direction pointing toward a front body portion of an amputee being fitted with exemplary universal socket 311. Exemplary universal socket 311 may be more comfortable to a user when exemplary universal socket 311 is positioned directly below an amputee's knee so that a portion of the amputee's leg above the knee can extend into a lower rim section 315 extending along rear rim section 313.

As shown in FIG. 5B, exemplary universal socket 411 may comprise first socket open end 25, wherein first socket open end 25 has an outer rim section 412 that is at a higher position relative to an inner rim section 413. In FIG. 5B, arrow F is used to show a direction pointing toward a front body portion of an amputee being fitted with exemplary universal socket 411. Exemplary universal socket 411 may be more comfortable to a user when exemplary universal socket 411 is positioned directly below an amputee's waist area so that no portion of exemplary universal socket 411 extends between an upper portion of the amputee's right leg and a crotch area of the amputee (i.e., gap area 415 is positioned between an upper portion of the amputee's right leg and a crotch area of the amputee).

### II. Method of Making Prosthetic Devices

The present invention further provides methods for making any of the above-described and herein described prosthetic devices. Methods for making any of the above-described prosthetic devices may comprise one or more of the following method steps:

(1) thermoforming (e.g., molding) one or more of the components (e.g., the universal socket, the rigid members, the foot, cushions, sections of the rigid member, etc.);

(2) cutting one or more slots (e.g., slot 24) into a universal socket component and/or cutting one or more rigid members so proper length;

(3) attaching various components to one another via mechanical fasteners (e.g., bolts, screws, etc.) or chemical layers (e.g., an adhesive layer);

(4) measuring the dimensions of an amputee's stump; and

(5) forming kits comprising at least one universal socket, one or more stump cushions sized to match the at least one universal socket, one or more rings or ring sections sized to match the at least one universal socket, one or more upper and lower rigid members, one or more artificial knee joints, and one or more artificial feet.

### III. Methods of Using Prosthetic Devices

The present invention is even further directed to methods of using any one of the above-described prosthetic devices. Methods of using any of the above-described prosthetic devices may comprise one or more of the following method steps:

(1) matching a given prosthetic device to an amputee's stump;

(2) inserting one or more stump cushions into a specifically matched universal socket (i.e., matched to a specific amputee);

(3) removing a release liner to expose pressure-sensitive adhesive along an outer surface of a ring section;

(4) attaching one of more ring sections to an inner surface of a universal socket;

(5) inserting the amputee's stump into the specifically matched universal socket until a stump end rests along an upper surface of a stump cushion;

(6) tightening the specifically matched universal socket around the amputee's stump via one or more tightening devices (e.g., straps, clamps, etc.);

(7) adjusting a length of a rigid member or rigid member components;

(8) adjusting a position of a lower rigid member within an upper rigid member so as to form a desire angle θ (see FIG. 3A); and

(9) training the amputee how to walk with the specifically matched prosthetic device.

While the specification has been described in detail with respect to specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

1. A prosthetic device comprising:
a universal socket operatively adapted and sized to receive a variety of stump sizes, said universal socket comprising:
a first socket open end sized to receive a user stump,
a second socket end opposite the first socket end,
at least two differently sized socket regions positioned between the first socket open end and the second socket end, said at least two differently sized socket regions comprising an upper socket region proximate the first socket open end and a lower socket region positioned between the upper socket region and the second socket end, wherein the upper socket region has an upper region cross-sectional area, the lower socket region has an lower region cross-sectional area, and the upper region cross-sectional area is greater than the lower region cross-sectional area, and
at least one slot extending from said first socket open end towards said second socket end.

2. The prosthetic device of claim 1, wherein said at least two differently sized socket regions comprises three differently sized socket regions comprising the upper socket region, the lower socket region, and a central socket region positioned between the upper socket region and the lower socket region, wherein the central socket region has a central region cross-sectional area, and the central region cross-sectional area is less than the upper region cross-sectional area and greater than the lower region cross-sectional area.

3. The prosthetic device of claim 1, wherein said universal socket further comprises:
concave grooves along inner side walls of said universal socket with at least one concave groove in each of said at least two differently sized socket regions.

4. (canceled)

5. The prosthetic device of claim 1, wherein said at least one slot extends into said lower socket region.

6. The prosthetic device of claim 1, wherein said at least one slot comprises two slots on opposite sides of said universal socket.

7. The prosthetic device of claim 1, wherein said universal socket further comprises:
one or more openings extending through a side wall of said universal socket from an outer surface to an inner surface of said universal socket.

8. The prosthetic device of claim 1, wherein said universal socket further comprises:
one or more tightening elements operatively adapted to tighten said universal socket onto a user's stump positioned within said universal socket.

9. The prosthetic device of claim 8, wherein said one or more tightening elements comprise one or more straps extending along an outer surface of said universal socket.

10. The prosthetic device of claim 9, wherein each strap has (i) a length so as to be capable of extending along an outer perimeter of said universal socket, and (ii) at least one fastening component that enables one portion of said strap to attach to another portion of said strap.

11-13. (canceled)

14. The prosthetic device of claim 1, wherein said universal socket has a tapered configuration.

15. The prosthetic device of claim 1, wherein said universal socket has a rim extending along said first socket open end, wherein said rim comprises at least two rim sections with at least one rim section being positioned above another rim section so as to form an uneven height of said universal socket.

16. The prosthetic device of claim 1, wherein said prosthetic device further comprises:
at least one stump cushion sized so as to be positionable within said universal socket.

17. (canceled)

18. The prosthetic device of claim 16, wherein said at least one stump cushion comprises multiple stump cushions with at least one stump cushion sized to fit within each of said at least two differently sized socket regions.

19-21. (canceled)

22. The prosthetic device of claim 1, wherein said prosthetic device further comprises:
at least one ring section, wherein each ring section (i) has an outer ring surface operatively adapted to contact an inner surface of said universal socket in a non-slip manner, (ii) an inner ring surface operatively adapted to contact an outer surface of a user's stump when positioned within said universal socket, and (iii) is sized so as to extend along an inner perimeter surface of said universal socket.

23-28. (canceled)

29. The prosthetic device of claim 22, wherein said at least one ring section comprises a tri-laminate comprising a silicone inner layer, a foam outer layer, and an adhesive layer positioned between said silicone inner layer and said foam outer layer.

30. The prosthetic device of claim 22, wherein said at least one ring section comprises a multi-layer configuration comprising a silicone inner layer, a foam outer layer, an adhesive layer positioned between said silicone inner layer and said foam outer layer, a pressure-sensitive adhesive layer positioned along at least a portion of an outer surface of said foam outer layer, and a removable release liner over said pressure-sensitive adhesive layer.

31. The prosthetic device of claim 1, wherein said prosthetic device further comprises:
an artificial foot; and
a rigid member extending from and connecting said second socket end to said artificial foot.

32. (canceled)

33. The prosthetic device of claim 31, wherein said rigid member is operatively adapted to (i) change in length so that a distance between said second socket end and said artificial foot is adjustable, and (ii) lock into a position so as to provide a fixed distance between said second socket end and said artificial foot.

34. (canceled)

35. The prosthetic device of claim 33, wherein said rigid member comprises (i) an upper rigid member comprising a tubular member having a first cross-sectional configuration, (ii) a lower rigid member comprising a solid or tubular member having a second cross-sectional configuration, said lower rigid member is sized so as to be insertable into an inner portion of said upper rigid member.

36. The prosthetic device of claim 35, wherein said upper rigid member further comprises at least two rows of holes extending along at least a lower outer perimeter of and encircling said upper rigid member with each hole extending from an outer surface into an interior volume of said upper rigid member, and a plurality of first fasteners operatively adapted to extend through said holes so as to contact and/or extend through an outer surface of said lower rigid member and secure said lower rigid member within said upper rigid member.

37-38. (canceled)

39. The prosthetic device of claim 35, wherein said prosthetic device further comprises a socket connector plate sized for placement within a lower region of said universal socket proximate said second socket end, a set of L-shape members, and a set of second fasteners, said socket connector plate, said set of L-shape members, and said set of second fasteners being operatively adapted to connect said upper rigid member to said second socket end of said universal socket.

40. (canceled)

41. The prosthetic device of claim 31, wherein said rigid member further comprises an artificial knee joint positioned along said rigid member.

42. The prosthetic device of claim 35, wherein said upper rigid member further comprises an artificial knee joint positioned along said upper rigid member.

43. The prosthetic device of claim 42, wherein said upper rigid member comprises an upper rigid member section, a lower rigid member section, a hinge member connecting said upper rigid member section to said lower rigid member section, and a tensioning member extending from said upper rigid member section to said lower rigid member section.

44. The prosthetic device of claim 43, wherein said hinge member comprises (i) a first hinge end attached to a rear surface of said lower rigid member section and (ii) a second hinge end attached to a front surface of said upper rigid member section.

45. The prosthetic device of claim 43, wherein said tensioning member (1) is positioned within a hollow volume of said lower rigid member section and (2) is connected to (i) said upper rigid member section, a portion of said hinge member connected to said upper rigid member section, or a plate connected to said portion of said hinge member connected to said upper rigid member section, and (ii) a location within said lower rigid member section.

46-50. (canceled)

51. The prosthetic device of claims 43, wherein one of said upper rigid member section and said lower rigid member section further comprises a first guide member and a second guide member along opposite side surfaces of said upper rigid member section or said lower rigid member section so as to provide guidance for restricted lateral movement of said upper rigid member section when said upper rigid member section moves relative to said lower rigid member section.

52-57. (canceled)
